(19) United States
(12) Patent Application Publication
(10) Pub. No.: US 2023/0310347 A1
(43) Pub. Date: Oct. 5, 2023

(54) BACTERICIDE

(71) Applicant: SHIRATORI PHARMACEUTICAL CO., LTD., Narashino-shi (JP)

(72) Inventors: Hiroshi YOSHINO, Narashino-shi (JP); Sho KAWATOBI, Narashino-shi (JP)

(73) Assignee: SHIRATORI PHARMACEUTICAL CO., LTD., Narashino-shi (JP)

(21) Appl. No.: 18/043,265

(22) PCT Filed: Sep. 1, 2021
(86) PCT No.: PCT/JP2021/032087

§ 371 Date: Feb. 27, 2023

(30) Foreign Application Priority Data
Sep. 1, 2020 (JP) 2020-146799

(51) Int. Cl. A61K 31/122; A61K 31/60; A61K 31/05; A61K 31/015; A61K 36/61; A61P 31/02

(52) U.S. Cl. CPC A61K 31/122; A61K 31/60; A61K 31/05; A61K 31/015; A61K 36/61; A61P 31/02

(57) ABSTRACT

Provided is a bactericide having an excellent bactericidal effect against Propionibacterium acnes. This bactericide contains the following components (A) and (B) as active ingredients:
(A) phenol derivative; and
(B) One or more ingredients selected from limonene, α-terpinene, γ-terpinene, terpinen-4-ol, and tea tree oil.

### TECHNICAL FIELD

The present invention relates to a bactericide.

### BACKGROUND ART

Propionibacterium acnes (Cutibacterium acnes) is a type of skin indigenous bacteria, and its proliferation is known to cause acne. In addition, Corynebacterium spp. are known to be a causative agent of armpit odor, and topical skin medications capable of killing these Cutibacterium acnes and Corynebacterium sp. have been developed. For example, it has been reported that phenolic bactericides such as Hinokitiol, Salicylic acid, and Isopropylmethylphenol etc. have bactericidal effects against Cutibacterium acnes (for example, Patent Document 1 and Non-Patent Document 1), but in recent years, further improvements in bactericidal effects have been required.

Meanwhile, tea tree (Melaleuca alternifolia) oil is known as an essential oil extracted from leaves of tea tree. It has been reported so far that tea tree oil contains terpinen-4-ol as the main ingredient and also contains 1,8-cineole, α-terpinene, γ-terpinene, α-terpineol, terpinolene and so forth and that it has fungicidal effects against fungi.

In addition, limonene is a monoterpene contained in essential oils of citrus fruits, such as orange, and has been reported to have fungicidal effects against fungi.

### PRIOR ART DOCUMENTS

### PATENT DOCUMENT

Patent Document 1: Japanese Unexamined Patent Publication 2007-77086

### Non-Patent Document

Non-Patent Document 1: Package insert, “Bifunight n,” Kobayashi Pharmaceutical Co., Ltd., December 2016.

### SUMMARY OF THE INVENTION

### Problem to be Solved by the Invention

Under the foregoing background, the present inventors examined the bactericidal effects of limonene, tea tree oil, and α-terpinene and γ-terpinene contained in tea tree oil, respectively, and found that any of the ingredients showed almost no bactericidal effects against Cutibacterium acnes. In addition, the bactericidal effects of terpinen-4-ol contained in tea tree oil against Cutibacterium acnes were insufficient.

The problem to be solved by the present invention is to provide a bactericide having superior bactericidal effects against Cutibacterium acnes.

### Means for Solving the Problem

As a result of intensive studies conducted by the present inventors, it was found that the bactericidal effects against Cutibacterium acnes were significantly enhanced when one or more ingredients selected from limonene, α-terpinene, γ-terpinene, terpinen-4-ol, and tea tree oil were combined with a phenol derivative typified by Hinokitiol, Salicylic acid, and Isopropylmethylphenol, and the inventors thus completed the present invention.

Accordingly, the present invention provides the following <1> to <8>.
<1> A bactericide comprising ingredients (A) and (B) as active ingredients,

wherein the ingredient (A) is a phenol derivative, and

the ingredient (B) is one or more ingredients selected from limonene, α-terpinene, γ-terpinene, terpinen-4-ol, and tea tree oil.
<2> The bactericide according to <1>, wherein the ingredient (A) is one or more phenol derivatives selected from Hinokitiol, Salicylic acid, and Isopropylmethylphenol.
<3> The bactericide according to <1> or <2>, wherein the ingredient (B) is one or more ingredients selected from limonene and γ-terpinene.
<4> The bactericide according to any one of <1>to <3>, wherein the ingredient (B) is limonene.
<5> The bactericide according to any one of <1>to <4>, wherein a content mass ratio of the ingredient (B) to ingredient (A) [(B)/(A)] is 0.2 or more and 2.5 or less.
<6> Use of a combination of ingredients (A) and (B) for manufacturing a bactericide,

wherein the ingredient (A) is a phenol derivative, and

the ingredient (B) is one or more ingredients selected from limonene, α-terpinene, γ-terpinene, terpinen-4-ol, and tea tree oil.
<7> Use of a combination of ingredients (A) and (B) for sterilization,

wherein the ingredient (A) is a phenol derivative, and

the ingredient (B) is one or more ingredients selected from limonene, α-terpinene, γ-terpinene, terpinen-4-ol, and tea tree oil.
<8> A sterilization method including steps using ingredients (A) and (B),

wherein the ingredient (A) is a phenol derivative, and

the ingredient (B) is one or more ingredients selected from limonene, α-terpinene, γ-terpinene, terpinen-4-ol, and tea tree oil.

### Effect of the Invention

The bactericide of the present invention has superior bactericidal effects against Cutibacterium acnes.

### MODE FOR CARRYING OUT THE INVENTION

### <Ingredient (A)>

In the present description, the term “phenol derivative” refers to a compound containing a phenol ring or tropolone ring in the molecule, and the phenol ring or tropolone ring in the molecule may have a substituent.

Phenol derivatives include, for example, Hinokitiol, Salicylic acid, Isopropylmethylphenol (4-isopropyl-3-methylphenol), phenol, cresol, chlorocresol, chloroxylenol, resorcinol, orthophenylphenol, orthophenylphenol salts (e.g., sodium orthophenylphenol) and so forth. It is noted that one of these may be used alone or two or more of these may be used in combination.

Among these, Hinokitiol, Salicylic acid, and Isopropylmethylphenol are preferable, and Salicylic acid is more preferable, in terms of bactericidal effect, safety, and availability.

As the phenol derivative, a commercially available product or a product obtained by synthesis according to a conventional method may be used.

### <Ingredient (B)>

The ingredient (B) of the present invention is one or more ingredients selected from limonene, α-terpinene, γ-terpinene, terpinen-4-ol, and tea tree (Melaleuca alternifolia) oil.

Limonene includes d-limonene, l-limonene, and mixtures of these, and preferably d-limonene.

In addition, tea tree oil means an essential oil extracted from leaves of tea tree, and usually contains 30% by mass or more of terpinen-4-ol, approximately 5 to 13% by mass of α-terpinene, and approximately 10 to 28% by mass of γ-terpinene.

When the ingredient (B) is to be contained in a bactericide, the ingredient (B) itself may be added, or an essential oil containing the ingredient (B) may be used. For example, essential oils containing limonene include orange oil, lemon oil, lemongrass oil, and so forth. In addition, while tea tree oil is given as one of essential oils containing α-terpinene, γ-terpinene, and terpinen-4-ol, it is preferable to use one that does not contain tea tree oil as the bactericide of the present invention in terms of bactericidal effect when α-terpinene and γ-terpinene are used as the ingredient (B). On the other hand, when terpinen-4-ol is used as the ingredient (B), it is preferable to use one that contains neither α-terpinene nor γ-terpinene as the bactericide of the present invention in terms of storage stability when coexisting with the ingredient (A).

Among the ingredients (B), limonene, α-terpinene, γ-terpinene and terpinen-4-ol are preferable, and limonene and γ-terpinene are more preferable in terms of bactericidal effect. On the other hand, limonene and terpinen-4-ol are preferable in terms of storage stability when coexisting with the ingredient (A). Among these ingredients (B), limonene is particularly preferable. When limonene is used as the ingredient (B), both superior bactericidal effects and superior storage stability can be achieved.

In addition, limonene, α-terpinene, γ-terpinene, terpinen-4-ol, and tea tree oil are known ingredients, and commercially available products or those obtained by synthesis according to conventional methods may be used.

The content mass ratio of the ingredient (B) to the ingredient (A) [(B)/(A)] is preferably 0.2 or more, more preferably 0.4 or more, still more preferably 0.5 or more, even more preferably 0.7 or more, and particularly preferably 0.9 or more, in terms of bactericidal effect and storage stability, and furthermore, preferably 2.5 or less, more preferably 1.8 or less, still more preferably 1.6 or less, even more preferably 1.4 or less, and particularly preferably 1.2 or less, in terms of bactericidal effect and storage stability. A specific range is preferably 0.2 or more and 2.5 or less, more preferably 0.4 or more and 2.5 or less, still more preferably 0.4 or more and 1.6 or less, and particularly preferably 0.9 or more and 1.2 or less.

It is noted that when the ingredient (B) is limonene, the bactericidal effects are further improved by setting the content mass ratio [(B)/(A)] to 0.2 or more and 1.6 or less. In addition, when the ingredient (B) is γ-terpinene, the bactericidal effects are further improved by setting the content mass ratio [(B)/(A)] to 0.2 or more and 2.5 or less, and when the ingredient (B) is tea tree oil, the bactericidal effects are further improved by setting the content mass ratio [(B)/(A)] to 0.4 or more and 2.5 or less.

Furthermore, as shown in Examples below, the combination of (A) phenol derivative and (B) one or more ingredients selected from limonene, α-terpinene, γ-terpinene, terpinen-4-ol, and tea tree oil has superior bactericidal effects against Propionibacterium acnes (Cutibacterium acnes). In addition, this combination also has superior bactericidal effects against a wide variety of bacteria other than Cutibacterium acnes, such as Corynebacterium xerosis, Staphylococcus aureus, Pseudomonas aeruginosa, and methicillin-resistant Staphylococcus aureus (MRSA). Here, the term “bactericidal/sterilization” as used in the present description refers to killing or destroying bacteria.

Bacteria other than Cutibacterium acnes are broadly classified into bacteria and fungi, and the bactericide of the present invention is suitable for killing bacteria. Bacteria include Gram-positive bacteria and Gram-negative bacteria.

Gram-positive bacteria include, for example, Propionibacterium or Cutibacterium spp. other than Cutibacterium acnes; Corynebacterium spp. such as Corynebacterium xerosis; Staphylococcus such as Staphylococcus aureus; Listeria spp.; Bacillus spp.; Alicyclobacillus spp.

Gram-negative bacteria include, for example, Escherichia spp. such as Escherichia coli; Salmonella spp.; Vibrio spp.; Pseudomonas spp. such as Pseudomonas aeruginosa; Acinetobacter spp. such as Acinetobacter baumannii; and Klebsiella spp. such as Klebsiella pneumoniae. In addition, the bactericide of the present invention has bactericidal effects against resistant bacteria such as methicillin-resistant Staphylococcus aureus (MRSA).

Among Cutibacterium acnes and bacteria other than Cutibacterium acnes, a combination of (A) a phenol derivative and (B) one or more ingredients selected from limonene, α-terpinene, γ-terpinene, terpinen-4-ol, and tea tree oil is suitable for killing Propionibacterium spp., Cutibacterium spp., Corynebacterium spp., Staphylococcus, Pseudomonas spp., and resistant bacteria such as MRSA, more suitable for killing Propionibacterium spp., Cutibacterium spp., Pseudomonas spp., and resistant bacteria such as MRSA, and particularly suitable for killing Propionibacterium spp. and Cutibacterium spp.

In addition, the bactericide of the present invention is useful for killing acne and armpit odor-causing bacteria such as Cutibacterium acnes and Corynebacterium spp. and can be used as a prophylactic and/or ameliorating agent for acne and a prophylactic and/or ameliorating agent for armpit odor.

Therefore, a combination of (A) a phenol derivative and (B) one or more ingredients selected from limonene, α-terpinene, γ-terpinene, terpinen-4-ol, and tea tree oil can be a bactericide, can be used for sterilization, and can be used for manufacturing bactericides.

Furthermore, the object of the above-mentioned “use” includes humans, non-human animals, specimens derived therefrom, articles, and so forth. For use in humans or non-human animals, it may be of therapeutic use (medical practice) or non-therapeutic use (non-medical practice). Examples of the above-mentioned articles include daily necessities, medical supplies, household electrical appliances/electric appliances, fittings (doors, window leaves, doorknobs, etc.) and so forth. In addition, the target article can be sterilized by spraying the article with a liquid type bactericide or by wiping the article with a sheet type bactericide.

It is noted that “non-therapeutic” is a concept that does not include medical practice, that is, it does not include methods of surgery, treatment, or diagnosis of humans, and more specifically, it does not include methods of performing surgery, treatment or diagnosis on humans by medical doctors, medical practitioners, or those under the direction of doctors.

In addition, the bactericide of the present invention can be used for drugs, quasi-drugs, or cosmetics which are effective for sterilization or as a material to be compounded in drugs, quasi-drugs, or cosmetics. In terms of making it suitable for drugs, quasi-drugs, and cosmetics, a topical use for the skin is preferable as a means of applying the bactericide of the present invention.

The bactericide of the present invention may be in a form of liquid, semi-solid, solid, or in a form in which a substrate such as a sheet type impregnated with the bactericide; in the case in which the bactericide of the present invention is a topical medication, specific forms thereof include ointments, creams, gels, topical liquids, lotions, sprays, topical aerosols, pump sprays, patches, tapes, poultices, topical solids, topical powders, liniments, and so forth. In addition, the bactericide of the present invention may be in a form of facial cleanser, skin toner, lotion, milky lotion, cream, mist, spray, face mask, body soap, shampoo, hair tonic, wet tissue, facial cleansing sheet, body sheet, and so forth.

The bactericide of the present invention may contain ingredients other than the ingredients (A) and (B), depending on the above-mentioned forms and applications. Such other ingredients include, for example, water, oils, surfactants, alcohols, perfumes, powders, chelating agents, antioxidants, UV inhibitors, thickeners, emulsion stabilizers, moisturizing agents, preservatives, pH adjusters, and so forth.

The content of the ingredient (A) is usually 0.00001 to 75% by mass with respect to the total mass of the bactericide of the present invention; however, when the bactericide of the present invention is a topical skin medication, it is preferably 0.00001 to 0.1% by mass, more preferably 0.00005 to 0.05% by mass, still more preferably 0.0001 to 0.01% by mass, even more preferably 0.0005 to 0.01% by mass, and particularly preferably 0.005 to 0.01% by mass.

The content of the ingredient (B) is usually 0.00001 to 75% by mass with respect to the total mass of the bactericide of the present invention; however, when the bactericide of the present invention is a topical skin medication, it is preferably 0.00001 to 0.1% by mass, more preferably 0.00005 to 0.05% by mass, still more preferably 0.0001 to 0.01% by mass, even more preferably 0.0005 to 0.01% by mass, and particularly preferably 0.005 to 0.01% by mass.

### EXAMPLES

The present invention will be described in detail below with reference to Examples, but the present invention is not limited to these Examples.

### Preparation Example 1

(Sample α1)

To Hinokitiol (natural Hinokitiol manufactured by Kiseitec Limited Company), a 10-fold amount (10 mL per 1 g of Hinokitiol) of sodium hydroxide aqueous solution (2 mol/L) was added, which was heated and dissolved in a water bath at 80° C., and then a 10000 μg/mL solution was obtained using purified water.

Meanwhile, to obtain 10000 μg/mL of tea tree oil (Lopovol Tea Tree manufactured by Vantage Specialty Ingredients, Inc., the same applies hereinafter), sodium dodecyl sulfate aqueous solution (0.1% by mass) was added to tea tree oil. 50 parts by mass of the resulting solution containing 10000 μg/ml of tea tree oil and 50 parts by mass of the above-mentioned solution containing 10000 μg/mL of Hinokitiol were mixed, and hydrochloric acid equimolar to the above-mentioned sodium hydroxide was added. Furthermore, purified water was added so that both Hinokitiol and tea tree oil were 100 μg/mL, and Sample α1(100 μg/mL of Hinokitiol+100 μg/mL of tea tree oil) was thereby prepared.

(Samples α2 to α5) The following Samples α2 to α5 were prepared in the same manner as Sample α1 except that tea tree oil was changed to limonene ((+)-Limonene manufactured by Tokyo Chemical Industry Co., Ltd., the same applies hereinafter), α-terpinene (manufactured by Tokyo Chemical Industry Co., Ltd.), γ-terpinene (manufactured by Tokyo Chemical Industry Co., Ltd.), and terpinen-4-ol (manufactured by Sigma-Aldrich Co. LLC), respectively.
Sample α2: 100 μg/mL of Hinokitiol+100 μg/mL of limonene
Sample α3: 100 μg/mL of Hinokitiol+100 μg/mL of α-terpinene
Sample α4: 100 μg/mL of Hinokitiol+100 μg/mL of γ-terpinene
Sample α5: 100 μg/mL of Hinokitiol+100 μg/mL of terpinen-4-ol

(Sample α6) To Salicylic acid (Salicylic acid (Japanese Standards of Quasi-drug Ingredients) manufactured by API Corporation), a 10-fold amount (10 mL per 1 g of Salicylic acid) of sodium hydroxide aqueous solution (2 mol/L) was added, and a 10000 μg/mL solution was obtained using purified water.

Meanwhile, to obtain 10000 μg/mL of tea tree oil, sodium dodecyl sulfate aqueous solution (0.1% by mass) was added to tea tree oil. 50 parts by mass of the resulting solution containing 10000 μg/mL of tea tree oil and 50 parts by mass of the above-mentioned aqueous solution containing 10000 μg/mL of Salicylic acid were mixed, and hydrochloric acid equimolar to the above-mentioned sodium hydroxide was added. Furthermore, purified water was added so that both Salicylic acid and tea tree oil were 100 μg/mL, and Sample α6 (100 μg/mL of Salicylic acid+100 μg/mL of tea tree oil) was thereby prepared.

(Sample α7)

Sample α7 (100 μg/mL of Isopropylmethylphenol+100 μg/mL of tea tree oil) was prepared in the same manner as Sample α6 except that Salicylic acid was changed to Isopropylmethylphenol (Isopropylmethylphenol manufactured by Osaka Kasei Co., Ltd.).

(Sample β1))

To Hinokitiol, a 10-fold amount (10 mL per 1 g of Hinokitiol) of sodium hydroxide aqueous solution (2 mol/L) was added, which was heated and dissolved in a water bath at 80° C., and then a 10000 μg/mL solution was obtained using purified water. Hydrochloric acid equimolar to the above-mentioned sodium hydroxide was added to this 10000 μg/mL solution. Furthermore, purified water was added so that Hinokitiol was 100 μg/ml, and Sample β1 (solution containing 100 μg/mL of Hinokitiol) was thereby prepared.

(Sample β2)

To Salicylic acid, a 10-fold amount (10 mL per 1 g of Salicylic acid) of sodium hydroxide aqueous solution (2 mol/L) was added, and a 10000 μg/mL solution was obtained using purified water. Furthermore, purified water was added so that Salicylic acid was 100 μg/mL, and Sample β2 (solution containing 100 μg/mL of Salicylic acid) was thereby prepared.

(Sample β3)

Sample β3 (solution containing 100 μg/mL of Isopropylmethylphenol) was prepared in the same manner as Sample β2 except that Salicylic acid was changed to Isopropylmethylphenol.

(Sample β4)

To obtain 10000 μg/mL of tea tree oil, sodium dodecyl sulfate aqueous solution (0.1% by mass) was added to tea tree oil. Furthermore, purified water was added so that tea tree oil was 100 μg/mL, and Sample β4 (solution containing 100 μg /mL of tea tree oil) was thereby prepared.

(Samples β5 to β8)

The following Samples β5 to β8 were prepared in the same manner as Sample β4 except that tea tree oil was changed to limonene, α-terpinene, γ-terpinene, and terpinen-4-ol, respectively.
Sample β5: solution containing 100 μg/mL of limonene
Sample β6: solution containing 100 μg/mL of α-terpinene
Sample β7: solution containing 100 μg/mL of γ-terpinene
Sample β8: solution containing 100 μg/mL of terpinen-4-ol

(Sample β9)

A 10-fold amount (10 mL per 1 g of Hinokitiol) of sodium hydroxide aqueous solution (2 mol/L) was added to Hinokitiol, which was heated and dissolved in a water bath at 80° C., and then a 10000 μg/mL solution was obtained using purified water.

Meanwhile, to Isopropylmethylphenol, a 10-fold amount (10 mL per 1 g of Isopropylmethylphenol) of sodium hydroxide aqueous solution (2 mol/L) was added, and a 10000 μg/mL solution was obtained using purified water. 50 parts by mass of the resulting solution containing 10000 μg/mL of Isopropylmethylphenol and 50 parts by mass of the above-mentioned solution containing 10000 μg/mL of Hinokitiol were mixed, and hydrochloric acid equimolar to the above-mentioned sodium hydroxide was added. Furthermore, purified water was added so that both Hinokitiol and Isopropylmethylphenol were 100 μg/mL, and Sample β9 (100 μg/mL of Hinokitiol+100 μg/mL of Isopropylmethylphenol) was thereby prepared.

[Experiment Example 1-1: Bactericidal Property (Propionibacterium acnes)]

Propionibacterium acnes (GAI 5419) was inoculated on a GAM agar medium (manufactured by Nissui Pharmaceutical Co., Ltd.) and precultured under anaerobic conditions at 35° C.±1° C. for 18 to 24 hours. A solution containing Propionibacterium acnes was prepared by diluting the solution to 10⁷ to 10⁸ bacteria/mL of Propionibacterium acnes with physiological saline.

Next, 10 mL of the samples obtained in Preparation Example 1 were inoculated with 0.1 mL of the above-mentioned solution containing Propionibacterium acnes. These were stored at room temperature for 30 minutes and then diluted 10-fold with an SCDLP agar medium (manufactured by Nihon Pharmaceutical Co., Ltd.) and neutralized. These were poured onto a GAM agar medium (manufactured by Nissui Pharmaceutical Co., Ltd.) according to the pour plate culture method, and anaerobically cultured at 35° C.±1° C. for 2 to 5 days. After that, the numbers of viable bacteria were determined from the numbers of colonies formed and the dilution factor. The results are shown in Tables 1 and 2. It is noted that physiological saline was used for the control in place of the samples.

TABLE 1

| | | Sample | | | | | | | |
|---|---|---|---|---|---|---|---|---|---|
| (μg/mL) | | α1 | α2 | α3 | α4 | α5 | α6 | α7 | Control |
| (A) | Hinokitiol | 100 | 100 | 100 | 100 | 100 | | | |
| | Salicylic acid | | | | | | 100 | | |
| | Isopropylmethylphenol | | | | | | | 100 | |
| (B) | Tea tree oil | 100 | | | | | 100 | 100 | |
| | Limonene | | 100 | | | | | | |
| | α-Terpinene | | | 100 | | | | | |
| | γ-Terpinene | | | | 100 | | | | |
| | Terpinen-4-ol | | | | | 100 | | | |

| | | | | | | | | |
|---|---|---|---|---|---|---|---|---|
| Number of viable bacteria | 0 | 20 | 10 | 0 | 0 | 0 | 0 | 440000 |
| (Propionibacterium acnes) | | | | | | | | |

TABLE 2

| | | Sample | | | | | | | | | |
|---|---|---|---|---|---|---|---|---|---|---|---|
| (μg/mL) | | β1 | β2 | β3 | β4 | β5 | β6 | β7 | β8 | β9 | Control |
| (A) | Hinokitiol | 100 | | | | | | | | 100 | |
| | Salicylic acid | | 100 | | | | | | | | |
| | Isopropylmethylphenol | | | 100 | | | | | | 100 | |
| (B) | Tea tree oil | | | | 100 | | | | | | |
| | Limonene | | | | | 100 | | | | | |
| | α-Terpinene | | | | | | 100 | | | | |
| | γ-Terpinene | | | | | | | 100 | | | |
| | Terpinen-4-ol | | | | | | | | 100 | | |

| | | | | | | | | | | |
|---|---|---|---|---|---|---|---|---|---|---|
| Number of viable bacteria | 220000 | 250 | 270000 | 360000 | 390000 | 470000 | 460000 | 200000 | 25000 | 440000 |
| (Propionibacterium acnes) | | | | | | | | | | |

As shown in Tables 1 and 2, Hinokitiol, Isopropylmethylphenol, and terpinen-4-ol had insufficient bactericidal effects against Propionibacterium acnes, and tea tree oil, limonene, α-terpinene, and γ-terpinene showed no bactericidal effects against Propionibacterium acnes.

In contrast, synergistically superior bactericidal effects were obtained in the cases in which an ingredient selected from Hinokitiol, Salicylic acid, and Isopropylmethylphenol was combined with an ingredient selected from tea tree oil, limonene, γ-terpinene, γ-terpinene, and terpinen-4-ol.

### [Preparation Example 2]

(Samples α8 to α23)

Samples α8 to α11 were prepared in the same manner as Sample α1 except that purified water was added so that Hinokitiol and tea tree oil had concentrations shown in Table 3.

Samples α12 to α15 were prepared in the same manner as Sample α2 except that purified water was added so that Hinokitiol and limonene had concentrations shown in Table 3.

Sample α16 was prepared in the same manner as Sample α3 except that purified water was added so that Hinokitiol and α-terpinene had concentrations shown in Table 4.

Samples α17 to α22 were prepared in the same manner as Sample α4 except that purified water was added so that Hinokitiol and γ-terpinene had concentrations shown in Table 4.

Sample α23 was prepared in the same manner as Sample α5 except that purified water was added so that Hinokitiol and terpinen-4-ol had concentrations shown in Table 4.

(Sample β10)

Sample β10 was prepared in the same manner as Sample β1 except that purified water was added so that Hinokitiol had a concentration shown in Table 5.

(Samples β11 to β15)

Samples β11 to β15 were prepared in the same manner as Samples β4 to β8 except that purified water was added so that the ingredients (B) had concentrations shown in Table 5.

[Experiment Example 1-2: Bactericidal Property (Propionibacterium acnes)]

The numbers of viable bacteria were determined in the same manner as Experiment Example 1-1 except that the samples obtained in Preparation Example 1 were changed to the samples obtained in Preparation Example 2.

The results are shown in Tables 3 to 5.

TABLE 3

| | | Sample | | | | | | | | |
|---|---|---|---|---|---|---|---|---|---|---|
| (μg/mL) | | α8 | α9 | α10 | α11 | α12 | α13 | α14 | α15 | Control |
| (A) | Hinokitiol | 50 | 50 | 40 | 30 | 50 | 50 | 50 | 40 | |
| | Salicylic acid | | | | | | | | | |
| | Isopropylmethylphenol | | | | | | | | | |
| (B) | Tea tree oil | 50 | 40 | 50 | 50 | | | | | |
| | Limonene | | | | | 50 | 40 | 30 | 50 | |
| | α-Terpinene | | | | | | | | | |
| | γ-Terpinene | | | | | | | | | |
| | Terpinen-4-ol | | | | | | | | | |

| | | | | | | | | | |
|---|---|---|---|---|---|---|---|---|---|
| Number of viable bacteria | 4300 | 9500 | 9800 | 18000 | 380 | 6000 | 10000 | 9800 | 440000 |
| (Propionibacterium acnes) | | | | | | | | | |

TABLE 4

| | | Sample | | | | | | | | |
|---|---|---|---|---|---|---|---|---|---|---|
| (μg/mL) | | α16 | α17 | α18 | α19 | α20 | α21 | α22 | α23 | Control |
| (A) | Hinokitiol | 50 | 50 | 50 | 50 | 50 | 40 | 30 | 50 | |
| | Salicylic acid | | | | | | | | | |
| | sopropylmethylphenol | | | | | | | | | |
| (B) | Tea tree oil | | | | | | | | | |
| | Limonene | | | | | | | | | |
| | α-Terpinene | 50 | | | | | | | | |
| | γ-Terpinene | | 50 | 40 | 30 | 20 | 50 | 50 | | |
| | Terpinen-4-ol | | | | | | | | 50 | |

| | | | | | | | | | |
|---|---|---|---|---|---|---|---|---|---|
| Number of viable bacteria | 2400 | 400 | 1200 | 5200 | 18000 | 3900 | 8400 | 1900 | 440000 |
| (Propionibacterium acnes) | | | | | | | | | |

TABLE 5

| | | Sample | | | | | | |
|---|---|---|---|---|---|---|---|---|
| (μg/mL) | | β10 | β11 | β12 | β13 | β14 | β15 | Control |
| (A) Hinokitiol | | 50 | | | | | | |
| | Salicylic acid | | | | | | | |
| | Isopropylmethylphenol | | | | | | | |
| (B) | Tea tree oil | | 50 | | | | | |
| | Limonene | | | 50 | | | | |
| | α-Terpinene | | | | 50 | | | |
| | γ-Terpinene | | | | | 50 | | |
| | Terpinen-4-ol | | | | | | 50 | |

| | | | | | | | |
|---|---|---|---|---|---|---|---|
| Number of viable bacteria | 290000 | 480000 | 440000 | 520000 | 400000 | 390000 | 440000 |
| (Propionibacterium acnes) | | | | | | | |

As shown in Tables 3 and 4, superior bactericidal effects were obtained against Propionibacterium acnes in the cases in which Hinokitiol was combined with an ingredient selected from tea tree oil, limonene, α-terpinene, γ-terpinene, and terpinen-4-ol even when the mass ratio was significantly changed. Moreover, it was found that, among the combinations of Hinokitiol and an ingredient selected from tea tree oil, limonene, α-terpinene, γ-terpinene, and terpinen-4-ol, the combination of Hinokitiol and an ingredient selected from limonene, α-terpinene, γ-terpinene, and terpinen-4-ol had higher bactericidal effects and that the combination of Hinokitiol and an ingredient selected from limonene and γ-terpinene had much higher bactericidal effects.

As shown in Table 5, even in the cases in which concentrations were 50 βg/mL, Hinokitiol and terpinen-4-ol had insufficient bactericidal effects against Propionibacterium acnes, and tea tree oil, limonene, α-terpinene, and γ-terpinene showed no bactericidal effects against Propionibacterium acnes as the results shown in Table 2.

### [Preparation Example 3]

(Samples α24 to α25)

Samples α24 and α25 were prepared in the same manner as Sample α1 except that purified water was added so that Hinokitiol and tea tree oil had concentrations shown in Table 6.

### [Experiment Example 2: Bactericidal Property (Corynebacterium spp.)]

Corynebacterium xerosis (NBRC 16721) was inoculated on a soybean-casein-digest agar medium (manufactured by Eiken Chemical Co., Ltd.) and precultured under aerobic conditions at 35° C.±1° C. for 2 days. A solution containing Corynebacterium sp. was prepared by diluting the bacteria with physiological saline to 10⁷ to 10⁸ bacteria/mL.

Next, 10 mL of the samples shown in Table 6 were inoculated with 0.1 mL of the above-mentioned solution containing Corynebacterium spp. These were stored at room temperature for 30 minutes and then diluted 10-fold with an SCDLP agar medium (manufactured by Nihon Pharmaceutical Co., Ltd.) and neutralized. These were poured onto an SCDLP agar medium (manufactured by Nihon Pharmaceutical Co., Ltd.) according to the pour plate culture method, and aerobically cultured at 35° C.±1° C. for 2 days. After that, the numbers of viable bacteria were determined from the numbers of colonies formed and the dilution factor. The results are shown in Table 6. It is noted that physiological saline was used for the control in place of the samples.

TABLE 6

| | | Sample | | | | | | | | |
|---|---|---|---|---|---|---|---|---|---|---|
| (μg/mL) | | α1 | α8 | α24 | α25 | α6 | α7 | β4 | β11 | Control |
| (A) | Hinokitiol | 100 | 50 | 10 | 5 | | | | | |
| | Salicylic acid | | | | | 100 | | | | |
| | Isopropylmethylphenol | | | | | | 100 | | | |
| (B) | Tea tree oil | 100 | 50 | 10 | 5 | 100 | 100 | 100 | 50 | |

| | | | | | | | | | |
|---|---|---|---|---|---|---|---|---|---|
| Number of viable bacteria | 0 | 0 | 50 | 60 | 0 | 0 | 330000 | 360000 | 210000 |
| (Corynebacterium xerosis) | | | | | | | | | |

[Experiment Example 3: Bactericidal Property (Pseudomonas aeruginosa)]

Pseudomonas aeruginosa (NBRC 13275) was inoculated on a nutrient agar medium (manufactured by Eiken Chemical Co., Ltd.) and precultured under aerobic conditions at 35° C.±1° C. for 18 to 24 hours.

A solution containing Pseudomonas aeruginosa was prepared by diluting the bacteria with purified water to 10⁷ to 10⁸ bacteria/mL.

Next, 10 mL of the samples shown in Table 7 were inoculated with 0.1 mL of the above-mentioned solution containing Pseudomonas aeruginosa. These were stored at room temperature for 30 minutes and then diluted 10-fold with an SCDLP agar medium (manufactured by Nihon Pharmaceutical Co., Ltd.) and neutralized. These were poured onto an SCDLP agar medium (manufactured by Nihon Pharmaceutical Co., Ltd.) according to the pour plate culture method, and aerobically cultured at 35° C.±1° C. for 2 days. After that, the numbers of viable bacteria were determined from the numbers of colonies formed and the dilution factor. The results are shown in Table 7. It is noted that purified water was used for the control in place of the samples.

TABLE 7

| | | Sample | | | | | | |
|---|---|---|---|---|---|---|---|---|
| (μg/mL) | | α1 | α8 | β1 | β10 | β4 | β11 | Control |
| (A) | Hinokitiol | 100 | 50 | 100 | 50 | | | |
| (B) | Tea tree oil | 100 | 50 | | | 100 | 50 | |

| | | | | | | | |
|---|---|---|---|---|---|---|---|
| Number of viable | 0 | 0 | 14000 | 21000 | 790000 | 820000 | 680000 |
| bacteria | | | | | | | |
| (Pseudomonas | | | | | | | |
| aeruginosa) | | | | | | | |

### [Experiment Example 4: Bactericidal Property (MRSA)]

Methicillin-resistant Staphylococcus aureus (MRSA (IID 1677)) was inoculated on a nutrient agar medium (manufactured by Eiken Chemical Co., Ltd.) and precultured under aerobic conditions at 35° C.±1° C. for 18 to 24 hours. A solution containing MRSA was prepared by diluting the bacteria with physiological saline to 10⁷ to 10⁸ bacteria/mL.

Next, 10 mL of the samples shown in Table 8 were inoculated with 0.1 mL of the above-mentioned solution containing MRSA. These were stored at room temperature for 30 minutes and then diluted 10-fold with an SCDLP agar medium (manufactured by Nihon Pharmaceutical Co., Ltd.) and neutralized. These were poured onto an SCDLP agar medium (manufactured by Nihon Pharmaceutical Co., Ltd.) according to the pour plate culture method, and aerobically cultured at 35° C.±1° C. for 2 days. After that, the numbers of viable bacteria were determined from the numbers of colonies formed and the dilution factor. The results are shown in Table 8. It is noted that physiological saline was used for the control in place of the samples.

TABLE 8

| | | Sample | | | | |
|---|---|---|---|---|---|---|
| (μg/mL) | | α1 | α8 | β10 | β11 | Control |
| (A) | Hinokitiol | 100 | 50 | 50 | | |
| (B) | Tea tree oil | 100 | 50 | | 50 | |

| | | | | | |
|---|---|---|---|---|---|
| Number of viable bacteria | 0 | 10 | 490 | 640000 | 550000 |
| (MRSA) | | | | | |

[Experiment Example 5: Bactericidal Property (Staphylococcus aureus subsp. aureus)]

The numbers of viable bacteria were determined in the same manner as Experiment Example 4 except that MRSA was changed to Staphylococcus aureus subsp. aureus (NBRC 12732) and that the samples shown in Table 9 were used as the samples. The results are shown in Table 9.

TABLE 9

| | | Sample | | | | Con- |
|---|---|---|---|---|---|---|
| (μg/mL) | | α1 | α8 | β4 | β11 | trol |
| (A) Hinokitiol | | 100 | 50 | | | |
| (B) | Tea tree oil | 100 | 50 | 100 | 50 | |
| Number of viable bacteria | | 0 | 0 | 340000 | 360000 | 300000 |
| (Staphylococcus aureus) | | | | | | |

### [Experiment Example 6-1: Storage Stability]

The storage stability of limonene alone and the storage stability of limonene mixed with Hinokitiol were evaluated.

Specifically, limonene was placed in a glass vial, which was hermetically sealed by tightening a septum with an aluminum cap.

Furthermore, 50 parts by mass of limonene and 50 parts by mass of Hinokitiol were mixed, placed in a glass vial different from the above-described glass vial, and hermetically sealed by tightening a septum with an aluminum cap. These two samples were stored at 60° C. for 7 days, and the area percentage of limonene in the glass vials was measured by GC (the instrument: GC-2010 Plus manufactured by Shimadzu Corporation; the detector: flame ionization detector; the column: DB-624 manufactured by Agilent Technologies Japan, Ltd.) immediately after the start of the experiment, after 1 day, after 4 days, and after 7 days. The results are shown in Table 10. The initial data (immediately after the start of the experiment) was the GC measurement result of limonene alone.

TABLE 10
Limonene residual ratio (area percentage (%))

| Hinokitiol | Absent | Present |
|---|---|---|
| Immediately after the start of the experiment | 96.85 | 96.85 |
| After 1 day | 95.67 | 96.49 |
| After 4 days | 95.45 | 96.57 |
| After 7 days | 94.43 | 96.41 |

### [Experiment Example 6-2: Storage Stability]

Regarding α-terpinene, γ-terpinene, and terpinen-4-ol, the storage stability of each of these alone and the storage stability of each of these mixed with Hinokitiol were evaluated.

Specifically, measurements were made in the same manner as Experiment Example 6-1 except that limonene was changed to α-terpinene, γ-terpinene, and terpinen-4-ol, respectively. It is noted that, regarding γ-terpinene, GC measurements were only made immediately after the start of the experiment, after one day, and after seven days. The results are shown in Tables 11 to 14.

TABLE 11
α-Terpinene residual ratio (area percentage (%))

| Hinokitiol | Absent | Present |
|---|---|---|
| Immediately after the start of the experiment | 89.95 | 89.95 |
| After 1 day | 85.27 | 82.68 |
| After 4 days | 81.54 | 77.11 |
| After 7 days | 78.45 | 70.49 |

TABLE 12
γ-Terpinene residual ratio (area percentage (%))

| Hinokitiol | Absent | Present |
|---|---|---|
| Immediately after the start of the experiment | 96.81 | 96.81 |
| After 1 day | 92.09 | 92.22 |
| After 7 days | 85.49 | 84.44 |

TABLE 13
Terpinen-4-ol residual ratio (area percentage (%))

| Hinokitiol | Absent | Present |
|---|---|---|
| Immediately after the start of the experiment | 96.94 | 96.94 |
| After 1 day | 92.86 | 97.13 |
| After 4 days | 84.09 | 96.83 |
| After 7 days | 74.45 | 96.52 |

As shown in Tables 10 to 13, limonene and terpinen-4-ol had superior storage stability when combined with a phenol derivative typified by Hinokitiol.

### [Experiment Example 6-3: Storage Stability]

Regarding terpinen-4-ol and γ-terpinene in tea tree oil, the storage stability of each of these in tea tree oil alone and the storage stability of each of these when tea tree oil was mixed with Hinokitiol were evaluated.

Specifically, the samples were stored in the same manner as Experiment Example 6-1 except that limonene was changed to tea tree oil, and the residual ratio of each of terpinen-4-ol and γ-terpinene in tea tree oil was measured by GC (the instrument: GC-2010 Plus manufactured by Shimadzu Corporation; the detector: a flame ionization detector; the column: DB-624 manufactured by Agilent Technologies Japan, Ltd.) immediately after the start of the experiment, after 1 day and after 7 days. The results are shown in Tables 14 and 15.

TABLE 14
Terpinen-4-ol residual ratio in tea tree oil (area percentage (%))

| Hinokitiol | Absent | Present |
|---|---|---|
| Immediately after the start of the experiment | 58.68 | 58.68 |
| After 1 day | 54.75 | 55.17 |
| After 7 days | 52.65 | 56.15 |

TABLE 15
γ-Terpinene residual ratio in tea tree oil (area percentage (%))

| Hinokitiol | Absent | Present |
|---|---|---|
| Immediately after the start of the experiment | 9.903 | 9.903 |
| After 1 day | 9.302 | 9.977 |
| After 7 days | 7.220 | 6.867 |

[Experiment Example 7: Bactericidal Property (Propionibacterium acnes)]

Sample α30 (100 μg/mL of Salicylic acid+100 μg/mL of α-terpinene) was prepared in the same manner as Sample α6 except that tea tree oil was changed to α-terpinene, and Sample α31 (100 μg/mL of Salicylic acid+100 μg/mL of limonene) was prepared in the same manner as Sample α6 except that tea tree oil was changed to limonene. Samples α32 and α33 were prepared in the same manner as Samples α30 and α31 except that purified water was added so that each of the ingredients had a concentration shown in Table 16.

The numbers of viable bacteria were determined in the same manner as Experiment Example 1-1 except that the samples obtained in Preparation Example 1 were changed to the above-mentioned Samples α30 to α33. The results are shown in Table 16.

TABLE 16

| | | Sample | | | | |
|---|---|---|---|---|---|---|
| (μg/mL) | | α30 | α31 | α32 | α33 | Control |
| (A) | Salicylic acid | 100 | 100 | 50 | 50 | |
| (B) | α-Terpinene | 100 | | 50 | | |
| | Limonene | | 100 | | 50 | |
| Number of viable bacteria | | 0 | 0 | 0 | 20 | 440000 |
| (Propionibacterium acnes) | | | | | | |

Furthermore, Sample β20 (100 μg/mL of Hinokitiol+100 μg/mL of linalool) was prepared in the same manner as Sample al except that tea tree oil was changed to linalool. The numbers of viable bacteria were determined in the same manner as Experiment Example 1-1 except that the samples obtained in Preparation Example 1 were changed to the above-mentioned Sample β20, but no bactericidal effects were observed (the number of viable Propionibacterium acnes: 540000).

1. A bactericide comprising:
an active ingredient (A) that is a phenol derivative; and
an active ingredient (B) that is one or more ingredients selected from limonene, α-terpinene, γ-terpinene, terpinen-4-ol, and tea tree oil.

2. The bactericide according to claim 1, wherein the active ingredient (A) is one or more phenol derivatives selected from the group consisting of Hinokitiol, Salicylic acid, and Isopropylmethylphenol.

3. The bactericide according to claim 1, wherein the active ingredient (B) is one or more ingredients selected from limonene and γ-terpinene.

4. The bactericide according to claim 1, wherein the active ingredient (B) is limonene.

5. The bactericide according to claim 1, wherein a content mass ratio of the active ingredient (B) to the active ingredient (A) [(B)/(A)] is 0.2 or more and 2.5 or less.

6. A method for manufacturing a bactericide comprising;
mixing an active ingredient (A) that is a phenol derivative; and
an active ingredient (B) that is one or more ingredients selected from the group consisting of limonene, α-terpinene, γ-terpinene, terpinen-4-ol, and tea tree oil.

7. A method for skin sterilization comprising;
applying to the skin an active ingredient (A) that is a phenol derivative; and
an active ingredient (B) that is one or more ingredients selected from the group consisting of limonene, α-terpinene, γ-terpinene, terpinen-4-ol, and tea tree oil.

8. A skin sterilization method comprising;
applying to the skin an active ingredient (A) with an active ingredient (B);
wherein the active ingredient (A) is a phenol derivative; and
the active ingredient (B) is one or more ingredients selected from the group consisting of limonene, α-terpinene, γ-terpinene, terpinen-4-ol, and tea tree oil.
